(19) United States
(12) Patent Application Publication
(10) Pub. No.: US 2007/0073259 A1
(43) Pub. Date: Mar. 29, 2007

(54) Absorbent article with dual gather leg cuff assemblies

(76) Inventors: Edward P. Erdman, West Chester, PA (US); Nicole DiVittore, West Chester, PA (US)

Correspondence Address: RATNERPRESTIA, P O BOX 980, VALLEY FORGE, PA, 19482-0980, US

(21) Appl. No.: 11/238,738

(22) Filed: Sep. 29, 2005

(51) Int. Cl. A61F 13/15

(52) U.S. Cl. 604/385.28

(57) ABSTRACT

A disposable absorbent article, which can be configured as a disposable diaper, a panty-type garment, or an adult incontinent product, includes a backsheet, an absorbent panel positioned on top of the backsheet, and a liquid permeable topsheet positioned on top of the absorbent panel. A pair of longitudinally extending, elasticized unitary leg cuff assemblies are positioned on respective opposite sides of a longitudinal centerline of the article. Each leg cuff assembly includes an outer sleeve having an upstanding inner gather and an upstanding outer gather.

### BACKGROUND OF THE INVENTION

The present invention relates generally to a disposable absorbent article which can be configured as a disposable diaper, training pant, or adult incontinent product, and more particularly to a disposable absorbent article having a pair of elasticized, upstanding unitary dual gather leg cuff assemblies configured for enhanced containment.

Disposable absorbent articles, such as disposable diapers and disposable pant-type garments (sometimes referred to as training pants) have found widespread acceptance for infant care. Garments of this nature are typically configured for a single use, with an absorbent panel or core of the construction ordinarily provided in an integrated structure including a liquid-pervious topsheet or facing layer, and a backsheet or backing layer having at least a portion which is liquid impervious. Adhesive coated fastening tabs, or hook-and-loop fasteners are typically provided on disposable diapers, while pant-type garments include side seams which can be readily torn for removal of the garment if soiled. Absorbent articles of the above nature are not only suitable for infants, but can be appropriately sized and configured for incontinence use by adults.

U.S. Pat. No. 5,403,301 discloses a disposable absorbent garment of the type suitable for infant care and for use by incontinent adults. In order to enhance the containment characteristics of the absorbent article disclosed in this patent, the construction includes elastication at laterally opposite side margins of the construction, thereby forming leg-encircling elasticized regions. Containment is further enhanced by the provision of an upstanding gather element, respectively positioned generally inwardly of each leg-encircling elasticized regions. This patent is hereby incorporated by reference.

Experience has shown that, in a disposable absorbent garment of the type disclosed in the above patent, the standing leg gather elements of the construction contribute substantially to the containment characteristics of the article. Nevertheless, there remains a need for improved absorbent garments and the standing leg gather elements of such garments. Accordingly, the present invention is directed to an improved absorbent garment such as a disposable absorbent article having improved dual gather unitary, elasticized leg cuff assemblies.

### SUMMARY OF THE INVENTION

According to one aspect of the invention, and according to one exemplary embodiment, a disposable absorbent article is provided with a backsheet, an absorbent panel positioned on top of the backsheet, and a topsheet positioned on top of the absorbent panel. A pair of longitudinally extending, elasticized leg cuff assemblies are positioned on respective opposite sides of a longitudinal centerline of the article, wherein each leg cuff assembly is configured to define an inner upstanding gather and an outer upstanding gather. The inner upstanding gather has a first height relative to the topsheet and the outer upstanding gather has a second height relative to the topsheet, wherein the first height is approximately 65% or less than the second height.

According to another aspect of the invention, and according to one exemplary embodiment, the disposable absorbent article is formed by orienting a pair of longitudinally extending, elasticized leg cuff assemblies on respective opposite sides of a longitudinal centerline of the article. An inner upstanding gather having a first height relative to a topsheet of the article and an outer upstanding gather having a second height relative to the topsheet are defined on each of the leg cuff assemblies. The first height is maintained at approximately 65% or less than the second height.

According to yet another aspect of the invention, and according to one exemplary embodiment, a disposable absorbent article is provided with an outer gather having an inner surface that is at an angle relative to a portion of the topsheet inboard of the outer gather that is less than or equal to about 90 degrees.

### BRIEF DESCRIPTION OF THE DRAWINGS

The invention is best understood from the following detailed description when read in connection with the accompanying drawings. It is emphasized that, according to common practice, the various features of the drawings are not to scale. On the contrary, the dimensions of the various features are arbitrarily expanded or reduced for clarity. Included in the drawings are the following figures:

FIG. 1 is a perspective view of an embodiment of a disposable absorbent article, configured as a training pant, embodying the principles of the present invention generally as it appears when being worn;

FIG. 2 is a top plan view of an embodiment of the present absorbent article generally in a form prior to final assembly;

FIG. 3 is a cross-sectional view of the absorbent article illustrated in FIG. 2, taken generally along lines 3-3 of FIG. 2;

FIG. 4 is a cross-sectional view similar to FIG. 3 illustrating a leg cuff assembly relative to the article body prior to attachment thereto;

FIG. 5 is a cross-sectional view of the absorbent article illustrated in FIG. 2, taken generally along lines 5-5 of FIG. 2; and

FIG. 6 is a cross-sectional view similar to FIG. 3 illustrating an alternative embodiment of the present invention.

### DETAILED DESCRIPTION OF THE INVENTION

This invention will now be described with reference to several embodiments selected for illustration in the drawings. It will be appreciated that the scope and spirit of the invention are not limited to the illustrated embodiments. It will further be appreciated that the drawings are not rendered to any particular proportion or scale.

Generally, a disposable absorbent article embodying the principles of the present invention can be configured as a disposable diaper, or a pant-type garment, for infants or small children, or can be appropriately sized and configured for use by incontinent adults. For enhancing the containment and fit characteristics of the article, the construction includes a pair of longitudinally extending, generally upstanding elasticized dual gather leg cuff assemblies. Each gather of each cuff assembly is provided with one or more elastically contractible elastic elements. The elastic elements are positioned within a unitary outer sleeve that defines both leg gathers of a given leg cuff assembly for distributing elastic contraction forces generated by the elastic elements over a substantial portion of the vertical extent of each leg cuff assembly.

The disposable absorbent article includes a backsheet preferably having at least a portion which is liquid impervious, and an absorbent panel positioned on top of the backsheet. The article further includes a liquid pervious topsheet positioned on top of the absorbent panel, with the topsheet being positionable generally adjacent to a wearer during use of the article.

In accordance with an embodiment of the present invention, a pair of longitudinally extending, elasticized dual gather leg cuff assemblies are positioned on respective opposite sides of a longitudinal centerline of the article. Each leg cuff assembly includes an outer sleeve joined to the topsheet of the article and defining the respective dual leg gathers. At least one elastic element is positioned within an upstanding portion of each leg gather, in a manner which desirably acts to distribute the elastic contraction forces generated by the elements along a substantial portion of the vertical extent of each leg gather. While elastication of the article is effected by the provision of the elastic elements of the leg gathers, it is presently preferred that the side marginal portions of the article are not otherwise elasticized. The resultant construction not only provides the desired containment and fit characteristics, with enhanced aesthetic appeal, but also facilitates high-speed manufacture for economical use.

Other features and advantages of aspects of the present invention will become readily apparent from the following detailed description of embodiments selected for illustration, the accompanying drawings, and the appended claims.

With reference now to the drawings, therein is illustrated a disposable absorbent article 10, illustrated as a disposable training pant. As used in the present disclosure, the term “absorbent article” is intended to refer to an article or garment which is worn by an individual for absorbing urine, fecal matter or other body fluids. It is understood that garments embodying the principles of the present invention can be appropriately sized for use by infants and children, and can further be sized for use by incontinent adults. Furthermore, the absorbent article includes fastenable diapers and the like in addition to the illustrated training pant.

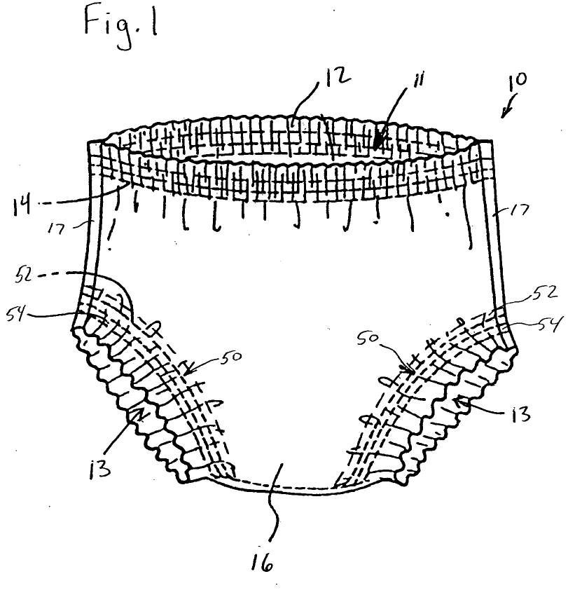

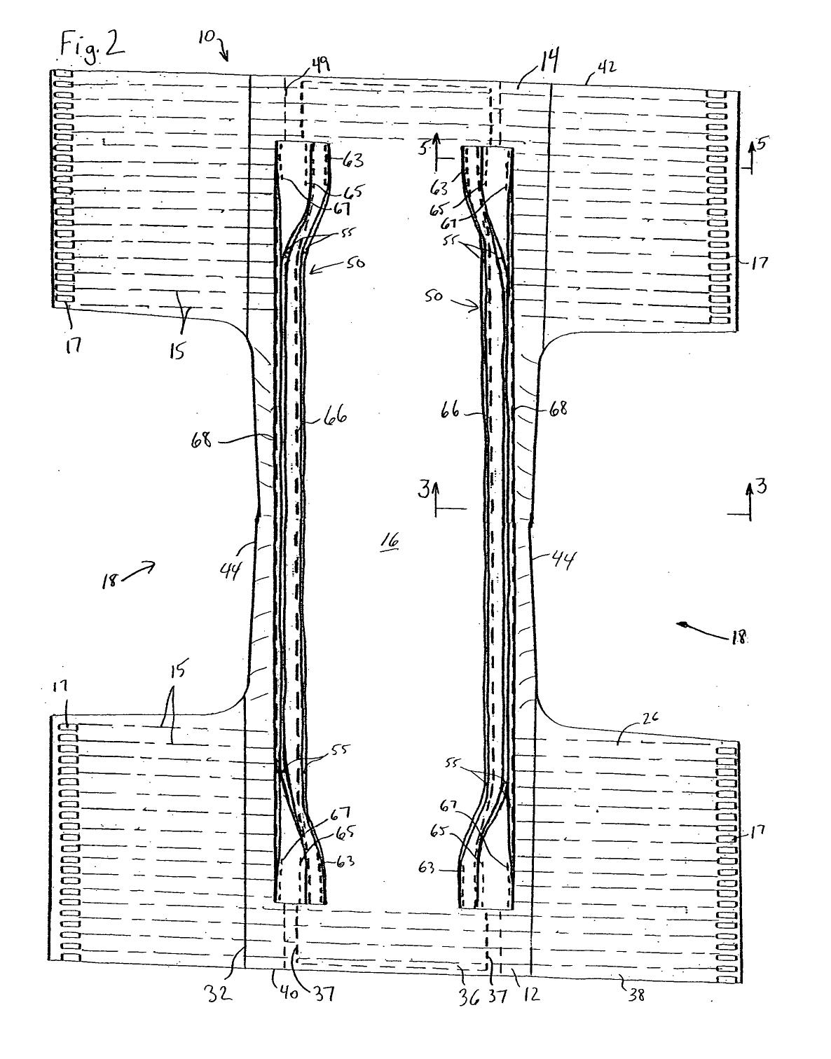

With particular reference to FIGS. 1 and 2, the absorbent article 10 includes a front waist portion 12, a rear waist portion 14, and a crotch portion 16 extending between the front and rear waist portions 12, 14. The crotch portion 16 of the garment generally corresponds to that portion which is positioned between the legs of a wearer during use, and as used herein, comprises between about one-third and three-fourths of the longitudinally central portion of the article. For enhanced fit and comfort, the disposable absorbent article 10 has a generally I-shaped, contoured configuration, with the crotch portion 16 being relatively narrow by virtue of the formation of leg cut-outs 18 at each of the side margins of the garment. In its final assembled form shown in FIG. 1, the front and rear waist portions 12 and 14 are attached or bonded at 17 such that the absorbent article 10 includes a waist opening 11 and a pair of leg holes 13. As seen in FIG. 2, the front and rear waist portions 12, 14 are each provided with a plurality of lateral elastic members 15. The elastic members 15 improve the article fit in the waist area.

With reference to FIGS. 2-5, the absorbent article 10 generally comprises a body-side liner or topsheet 32, a liquid absorbent structure or core 36, an outer cover or backsheet 38, and a pair of opposed dual gather leg cuff assemblies 50.

The topsheet 32 is arranged to face toward the body of the user when the absorbent article 10 is in place, with the backsheet 38 facing away from the wearer. The topsheet 32 is superimposed over the backsheet 38, with the absorbent core 36 interposed therebetween. A hydrophilic liquid distributing transfer layer 34 may be provided between the topsheet 32 and the absorbent core 36 to manage, transport, accommodate and/or direct urine or other body fluid received from the topsheet “target” or fluid “insult” zone (i.e., the area at which the body fluid(s) gain(s) ingress into the article) into the absorbent core 36.

The topsheet 32 may be coextensive in size and shape with the backsheet 38, or alternatively, may only cover a portion of the backsheet 38 as in the illustrated embodiment. In the present embodiment, the backsheet 38 extends beyond the topsheet 32 in the waist portions 12 and 14 to define lateral extensions 26. The backsheet 38, alone or in combination with the topsheet 32, defines the extents of the article 10 including a front edge 40, a back edge 42, and a pair of side edges 44. The front and back edges 40 and 42 form the front and rear waist sections 12 and 14, respectively of the article 10 when it is worn. Each side edge 44 includes a central, cut-out to define a respective leg cut out 18. The crotch portion 16 of the article is located between the leg cut-outs 18. The topsheet 32 and/or backsheet 38 can be any suitable shape and dimensions for other designs or constructions, as will be clear to those skilled in the art.

The topsheet 32 is bonded to the backsheet 38 around its periphery, with the absorbent material core 36 and the hydrophilic liquid distributing transfer layer 34 interposed therebetween. The backsheet 38 and topsheet 32 can be joined together in any suitable manner, e.g., by adhesive bonding. The adhesives can be applied in any manner such as by spraying, slot-coat extrusion, printing, or the like. The applied adhesive can be in any desired configuration or design, such as continuous or discontinuous beads, continuous or discontinuous swirls, meltblown patterns, spray patterns, or the like. Alternatively, the joining of layers and structures can be accomplished by heat sealing, ultrasonic bonding, or the like.

The backsheet 38 or cover is preferably formed of a laminated sheet of a non-woven material and a film 49 (with the non-woven side positioned as the outermost layer). The film layer 49 is centered in the article and extends the length of the article, i.e., from the front edge 40 to the back edge 42 and for a substantial width of the crotch portion 16 as best seen in FIG. 2. The material of the backsheet 38 should be hydrophobic, soft in texture, and strong in tensile strength. One particularly suitable material is a spunbond-meltblow-spunbond (SMS) web, available from AVGOL Nonwoven Industries LTD., Holon, Israel. The spunbond layer is made of polypropylene fibers. Such composites provide the dual advantages of liquid barrier properties of film along with a soft, warm outer fabric texture. The non-woven outer cover can also be made of other suitable cloth-like materials, e.g., spun-bond or thermal-bond non-woven web made of either polypropylene, polyethylene, polyester, bi-component fibers (polyethylene/polypropylene or polyethylene/polyester), or any combinations of these fibers. Various multiple layer configurations or fiber denier variations may be used. Another example includes hydro-entangled non-woven webs, which may contain some cotton and/or rayon fibers blending in with thermal-plastic fibers. Cellulose fibers can also be blended in at small percentages to reduce cost. Still another example is a non-woven outer-cover made of stretchable or elastic materials, such as elastomeric composites of non-woven(s) and elastic membranes or a single layer of elastic material. The elastomeric composite can comprise an inner layer of pre-stretched extruded elastic film sandwiched between and attached to a pair of non-woven webs. The non-woven webs may consist of spun-bond web, thermal-bond web, or a combination of the two. Preferably, the elastic film is made of synthetic rubber and the non-woven made of spun-bond polypropylene.

Other materials for forming the backsheet 38 may include polypropylene films, co-extruded films (polyethylene and ethylene vinyl acetate), co-polymer films (polyethylene/polypropylene), and polylaminates (polypropylene nonwoven and polyethylene film). Still another example is a film made of a “breathable” microporous polyethylene. Suitable breathable films are available from Exxon Chemical Company, Buffalo Grove, Ill. This material allows water vapor to pass through it over time, while being impervious to liquids. The water vapor transmission rate may range from 200-2000 grams per square meter per 24-hour period.

In order to enable waste to quickly and efficiently pass through the topsheet 32, the topsheet 32 is preferably liquid permeable (e.g., hydrophilic). In particular, the topsheet may be selected from a variety of textile-like films and fabrics. Suitable fabrics include non-woven materials that are pervious to liquid, soft and pliable. Preferred non-woven materials include spun-bonded polypropylene; spunbonded polyethylene; thermally bonded webs of staple fibers preferably polypropylene or sheath/core bi-component fibers having a core of polyester or polypropylene and a sheath of polyethylene. To enhance the fluid control properties of the aforementioned liners, surfactants or wetting agents typified by Triton X-100 and X-102 available from Rohm & Haas Company of Philadelphia, Pa. may be applied to the fluid receiving zones of the liner selectively having the outer zones untreated to reduce migration of excreted fluid such as urine into the outer diaper regions leading to diaper leakage. If desired, the topsheet 32 may be formed of a liquid impermeable material having plural apertures or pores extending therethrough so as to make the material liquid permeable.

The absorbent core 36 is centered in the absorbent article 10 and extends from close to the front waist edge 40 to close to the back waist edge 42. The core 36 can be made up of any suitable absorbent material, as well as combinations of different types of absorbent materials. For example, the absorbent core 36 may be formed of a mixture of pulp fluff and SAP. Examples of SAP include polyacrylamides, polyvinyl alcohol, polyacrylates, various grafted starches, and the like. A desired super absorbent material is a cross-linked polysodium acrylate, which can be purchased from Chemdal Corporation, Palatine, Ill., under the trademark ASAP 2260. The super absorbent materials can be in various geometric forms, such as various shaped particles, fibers, foams, and layers.

Moreover, the core 36 can be of any shape and can be a single, integral absorbent structure, or can comprise a plurality of individual separate absorbent structures and/or absorbent materials that are operably assembled together. It can also consist of air-laid non-woven web that contains super-absorbent particles and/or super-absorbent fibers, polymeric binder and cellulose pulp fibers. The absorbent core is centered along the transverse direction and registered in the machine, or longitudinal, direction within the article's chassis.

Referring to FIGS. 2-5, the absorbent article 10 also includes a pair of dual leg gather leg cuff assemblies 50 to provide leakage control in the crotch region. Each leg cuff assembly 50 extends longitudinally adjacent a respective longitudinal edge 37 of the absorbent core 36. Each leg cuff assembly 50 includes an upstanding inner gather 52, an upstanding outer gather 54 and a base portion 56 therebetween. The inner and outer gathers 52 and 54 and the base portion 56 are defined by an outer sleeve 60 of nonwoven hydrophobic material. The hydrophobic nonwoven material has a water repellency value of about 80 mm or more per ISO811-1981. A suitable fabric can comprise spunbonded polypropylene nonwoven fabric having a basis weight in the range of about 0.03 to 0.8 ounces per square yard and a bond area in the range of 7% to 20%, with a basis weight of about 0.5 to 0.6 ounces per square yard, and an 18% bond area being particularly preferred. When untreated, this material exhibits the desired degree of hydrophobicity. One commercially available material of this type is available from Fiberweb of America, Greenville, S.C., under the product designation Unicorn Celestra.

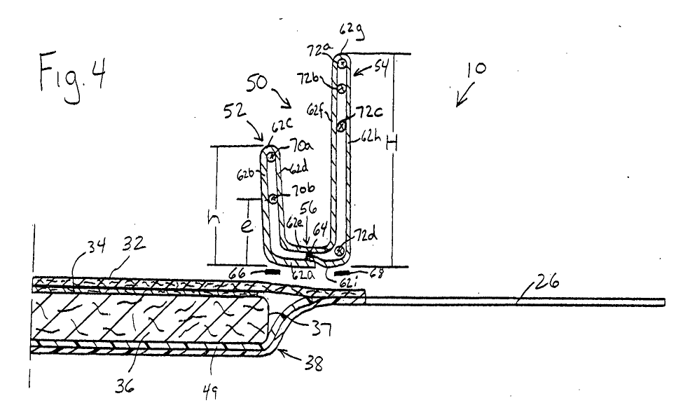

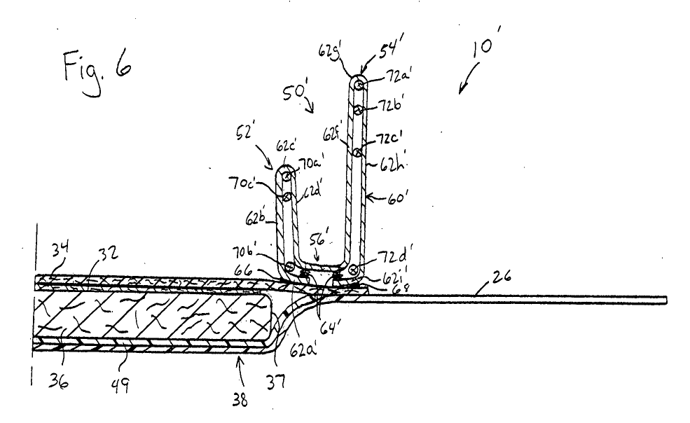

Referring to FIG. 4, the sleeve 60 includes an inner gather contact surface 62a and an outer gather contact surface 62i that together with an intermediate portion 62e define the base portion 56. The contact surfaces 62a and 62i may overlap, as illustrated, or may be spaced from one another as illustrated in the embodiment of the absorbent article 10′ in FIG. 6. One or more stitches, adhesives or other bonds, as indicated at 64 (64′ in FIG. 6) may be provided between the inner and outer contact surfaces 62a and 62i (62a′ and 62i′ in FIG. 6) and the intermediate portion 62e (62e′ in FIG. 6) to unify the leg cuff assembly 50 prior to attachment to the article chassis.

The sleeve 60 extends from the inner gather contact surface 62a to define the upstanding inner gather 52. The inner gather 52 is defined by inner extending portion 62b, transition portion 62c and outer extending portion 62d. The inner and outer extending portions 62b and 62d and the transition portion 62c extend about elastic elements 70a and 70b to cause the inner leg gather 52 to stand up relative to the topsheet 32, as will be described in more detail hereinafter.

The intermediate portion 62e extends between the inner gather 52 and the outer gather 54. The outer gather 54 is defined by inner extending portion 62f, transition portion 62g and outer extending portion 62h. The inner and outer extending portions 62f and 62h and the transition portion 62g extend about elastic elements 72a, 72b, 72c and 72d to define the outer gather 54 to cause the outer leg gather 54 to stand up relative to the topsheet 32, as will be described in more detail hereinafter. As illustrated in FIG. 4, the inner gather 52 has an upstanding height h and the outer gather 54 has an upstanding height H. The inner gather height h is preferably in a range of between approximately 35-70% of the outer gather height H; and more preferably in a range of approximately 50-65% of the outer gather height H.

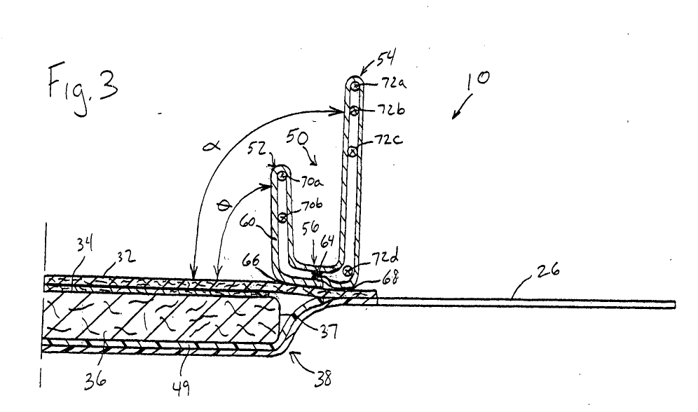

The base portion 56 is bonded to the article chassis adjacent a longitudinal edge 37 of the absorbent core 36. In the present embodiment, as illustrated in FIG. 3, the leg cuff assembly 50 is positioned such that the inner extending portion 62b of the inner gather 52 is inboard, i.e. toward the article centerline, of the respective longitudinal edge 37 of the absorbent core 36. However, as illustrated in the embodiment of the absorbent article 10′ shown in FIG. 6, the leg cuff assemblies 50′ may be positioned such that the inner extending portion 62b′ of the inner gather 52′ is positioned outboard, i.e., away from the article centerline, of the respective longitudinal edge 37 of the absorbent core 36.

The leg cuff assembly 50 is retained relative to the article chassis via one or more attachment bonds. In the present embodiment, an attachment bond 66 is provided between the inner contact surface 62a and the topsheet 32 and an attachment bond 68 is provided between the outer contact surface 62i and the topsheet 32. The bonds 66 and 68 are represented by dashed lines in FIG. 2. The bonds 66 and 68 may be provided utilizing various methods, including sonic or ultrasonic bonding, suitable adhesive, heat sealing or any other suitable bonding method.

Each of the longitudinally extending leg cuff assemblies 50 is elasticized to cause the leg gathers 52 and 54 to stand relative to the article chassis and to provide the desired containment and fit characteristics for the disposable absorbent article 10. Each inner gather 52 includes at least a first distal elastic member 70a adjacent the transition portion 62c of the inner gather 52 and each outer gather 54 includes at least a first distal elastic member 72a adjacent the transition portion 62g of the outer gather 54. Each of these elastic members 70a and 72a extend a substantial longitudinal length of the respective gathers 52 and 54, but may be terminated short of the longitudinal ends of the gathers 52, 54, as indicated at 55 in FIG. 2, to reduce longitudinal contraction in the front and rear waist portions 12 and 14. Securement of the ends of the leg gathers 52 and 54 to the article chassis in each waist portion 12, 14 maintains the elastic members 70a and 72a in an elastically stretched condition such that the elastics 70a and 72a cause the respective leg gathers 52 and 54 to stand upwardly relative to the topsheet 32. The elastic members 70a and 72a are initially elastically stretched, for example, approximately 200% to 350% relative to their relaxed length. This initial stretched condition maintains the gathers 52 and 54 in the standing position. A worn stretched condition allows the gathers 52 and 54 to conform to the user's anatomy.

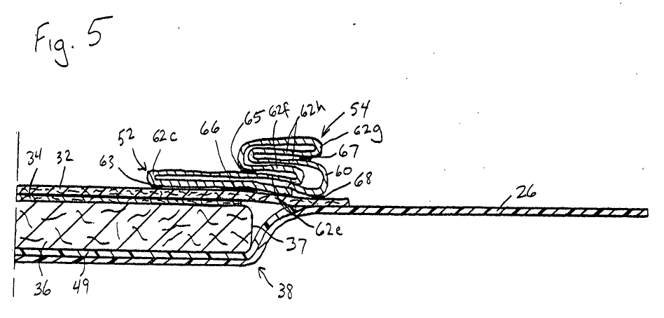

Referring to FIGS. 2 and 5, securement of each leg cuff assembly 50 in the waist portions 12 and 14 will be described. In the present embodiment, the distal end of inner gather 52 adjacent the transition portion 62c is secured, as indicated by bond 63, inboard of the inner gather bond 66. As illustrated in FIG. 2, the bond 63 is provided along only portions of the longitudinal ends of the inner gathers 52. Approximately 10% of the total length of each leg cuff assembly 50 is secured inwardly to the associated topsheet 32 at each end of each leg cuff assembly, i.e., 20% of the entire length is attached to the topsheet 32. The inward securement of the ends of each inner gather 52 causes each inner gather 52 to extend at an angle θ of less than or equal to about 90 degrees relative to an inboard portion of the topsheet 32 in the initial stretched condition, i.e., in an assembled, but unworn condition. This angle θ of the inner gathers 52 assists in retaining expelled body fluids within the insult zone above the absorbent core 36.

As shown in FIG. 5, the outer gather 54 is folded upon itself in a zigzag manner and secured in this position. In the first fold, the inner extending portion 62f is secured at bond 65 to the transition portion 62e. The bond 65 is laterally positioned proximate to the inner gather bond 66. The outer gather 54 is then folded back upon itself and the outer extending portion 62h is secured to itself at bond 67. The bond 67 is laterally proximate to the outer gather bond 68 such that the outer gather transition portion 62g is laterally proximate to the outer gather bond 68. This configuration maintains the outer gather 54 upstanding at an angle α of less than or equal to about 90 degrees relative to an inboard portion of the topsheet 32 in the initial stretched condition. This angle a of the outer gathers 54 provides comfort to the wearer while still assisting in retaining material which may manage to bypass the inner gather 52. Prior art cuffs have generally provided the outer gather at an outward angle, i.e. greater than 90 degrees, with a resultant outward leak path for materials that manage to bypass an inner cuff.

To further enhance the containment and fit provided by the upstanding leg gathers 52 and 54, each gather 52 and 54 may be provided with more than the one elastic member. Referring to FIG. 3, the inner gather 52 may have one or more additional elastic elements 70b. In the present embodiment, the elastic element 70b is positioned at a height e that is greater than 50% of the inner gather height h. This configuration of the elastic members 70a and 70b assists in maintaining the angle θ and to further retain material in the insult zone. Alternatively, the embodiment of the absorbent article 10′ illustrated in FIG. 6 illustrates the elastic element 70b′ proximate to the base portion 56′. Positioning of the elastic element 70b′ proximate to the base portion 56′ assists in controlling the elastically contractible gathering force asserted from each leg cuff assembly 50 on the article chassis. Additional elastic elements may be positioned between the elastic elements 70a′ and 70b′ as indicated by elastic element 70c′ in FIG. 6.

The outer gather 54 illustrated in FIG. 3 has four elastic members 72a, 72b, 72c and 72d, however, fewer or more elastic members may be provided. The elastic members 72a, 72b and 72c toward the distal end of the outer gather 54 assist to maintain the outer gather 54 upstanding at the desired angle α. The lower elastic element 72d assists in controlling the elastically contractible gathering force asserted from each leg cuff assembly 50 on the article chassis.

In both the inner and outer gathers 52; 52′ and 54; 54′, it is preferable that the distal half of each gather 52; 52′ and 54; 54′ has a higher total decitex than the proximal half thereof. For example, in the embodiment illustrated in FIG. 6, the elastic members are positioned, when the sleeve 60′ is flat prior attachment to the article, at the following positions starting from the outer gather transition 62g′: elastic element 72a′ is positioned at the transition 62g′; elastic element 72b′ is positioned approximately 10 mm from the transition 62g′; elastic element 72c′ is positioned approximately 20 mm from the transition 62g′; elastic element 72d′ is positioned approximately 39 mm from the transition 62g′; elastic element 70b′ is positioned approximately 54 mm from the transition 62g′; elastic element 70c′ is positioned approximately 69 mm from the transition 62g′; and elastic element 70a′ is positioned approximately 79 mm from the transition 62g. As such, elastic elements 70a′ and 70c′ are provided in the distal half of inner gather 52′ while only elastic element 70b′ is in the proximal half of inner gather 52′. Similarly, elastic elements 72a′, 72b′ and 72c′ are provided in the distal half of outer gather 54′ while only elastic element 72d′ is in the proximal half of outer gather 52′. The decitex of each elastic element 70a,b; 70a′,b′,c′ and 72a-d; 72a′-d′ does not have to be equal, but it is preferable to maintain a larger total decitex in the distal half of each gather 52; 52′ and 54; 54′.

The elastic elements 70a,b; 70a′,b′,c′ and 72a-d; 72a′-d′ can have various configurations. For example, the elastic elements 70a,b and 72a-d may be longitudinally extending elastic, e.g., LYCRA 800-1100 decitex, threads or strands. The strands may be obtained from E.I. DuPont de Nemours and Company, Wilmington, Del. The elastic members are configured such that the total decitex of all the elastic elements 70a,b; 70a′,b′,c′ and 72a-d; 72a′-d′ is between about 4000 to 7000 relaxed. The outer gather 54; 54′ will have a total decitex that is equal to or greater than the total decitex of the inner gather. Upon assembly of the article 10; 10′, the elastic elements 70a,b; 70a′,b′,c′ and 72a-d; 72a′-d′ will be stretched approximately 200% to 350% relative to the relaxed length. The elastic elements 72a-d; 72a′-d′ of the outer gather portion are preferably elongated to at least the same percentage as the elastic elements 70a,b; 70a′,b′,c′ of the inner gather 52; 52′.

The elastic elements 70a,b; 70a′,b′,c′ and 72a-d; 72a′-d′ are held in place within the sleeve 60, 60′ by a suitable elastic adhesive or other bonding method. The elastic member bonding may be continuous, intermittent at regular or irregular distances, or only provided at the ends of the elastic members. Other arrangements can be used to elasticize the leg gathers besides threads. For example, in lieu of plural longitudinally extending elastic threads, multiple strands of elastic material can be arranged in other orientations, intersecting, diagonal, or any combination thereof, or can be a film or laminate of various types of elastomeric material.

While leg cuff assemblies 50; 50′ have been illustrated as being generally linear and in parallel relationship to each other and to the centerline of the absorbent article 10, 10′, it is within the purview of the present invention to position the leg cuff assemblies in a curved or otherwise non-parallel configuration.

From the foregoing, it will be observed that numerous modifications and variations can be effected without departing from the true spirit and scope of the novel concept of the present invention. It is to be understood that no limitation with respect to the specific embodiments illustrated herein is intended or should be inferred. The disclosure is intended to cover, by the appended claims, all such modifications as fall within the scope of the claims.

What is claimed:

1. A disposable absorbent article, comprising:
a backsheet;
an absorbent panel positioned on top of said backsheet;
a topsheet positioned on top of said absorbent panel;
a pair of longitudinally extending, elasticized leg cuff assemblies positioned on respective opposite sides of a longitudinal centerline of said article, wherein each said leg cuff assembly is configured to define an inner upstanding gather and an outer upstanding gather, the inner upstanding gather having a first height relative to the topsheet and the outer upstanding gather having a second height relative to the topsheet, wherein the first height is approximately 65% or less than the second height.

2. A disposable absorbent article in accordance with claim 1 wherein each outer gather has an inner surface that is at an angle relative to a portion of the topsheet inboard of the outer gather that is less than or equal to about 90 degrees when the respective leg cuff assembly is in an initial stretched condition.

3. A disposable absorbent article in accordance with claim 2 wherein each inner gather has an inner surface that is at an angle relative to a portion of the topsheet inboard of the inner gather that is less than or equal to about 90 degrees when the respective leg cuff assembly is in the initial stretched condition.

4. A disposable absorbent article in accordance with claim 1 wherein the inner and outer gathers of each leg cuff assembly comprises a sleeve of hydrophobic material.

5. A disposable absorbent article in accordance with claim 4 wherein each sleeve includes a portion defining the inner gather, a portion defining the outer gather and a portion therebetween defining a base portion.

6. A disposable absorbent article in accordance with claim 5 wherein the sleeve portion defining the inner gather encloses a first inner elastic strand positioned adjacent a distal end thereof and the sleeve portion defining the outer gather encloses a first outer elastic strand positioned adjacent a distal end thereof.

7. A disposable absorbent article in accordance with claim 6 wherein the sleeve portion defining the inner gather encloses a second inner elastic strand positioned adjacent a proximal end thereof and the sleeve portion defining the outer gather encloses a second outer elastic strand positioned adjacent a proximal end thereof.

8. A disposable absorbent article in accordance with claim 6 wherein the sleeve portion defining the inner gather encloses a second inner elastic strand positioned closer to the distal end than a proximal end thereof and the sleeve portion defining the outer gather encloses a second outer elastic strand positioned closer to the distal end than a proximal end thereof.

9. A disposable absorbent article in accordance with claim 5 wherein the sleeve portion defining the inner gather encloses a plurality of inner elastic strands and the sleeve portion defining the outer gather encloses a plurality of outer elastic strands and wherein the plurality of outer elastic strands have a total decitex equal to or greater than a total decitex of the plurality of inner elastic strands.

10. A disposable absorbent article in accordance with claim 5 wherein the sleeve portion defining the inner gather encloses a plurality of inner elastic strands and the sleeve portion defining the outer gather encloses a plurality of outer elastic strands and wherein each of the outer elastic strands has an elongation percentage equal to or greater than an elongation percentage of the inner elastic strands.

11. A disposable absorbent article in accordance with claim 5 wherein each sleeve extends longitudinally between first and second ends and the sleeve portion defining the inner gather encloses a plurality of inner elastic strands and the sleeve portion defining the outer gather encloses a plurality of outer elastic strands and wherein each of the inner and outer elastic strands is bonded to the respective sleeve portion between the first and second ends.

12. A disposable absorbent article in accordance with claim 11 wherein each of the inner and outer elastic strands is free of bonding adjacent the first and second ends of the sleeve.

13. A disposable absorbent article in accordance with claim 5 wherein the base portion of each sleeve is bonded to the topsheet along an inner bond edge adjacent the inner gather and an outer bond edge adjacent the outer gather.

14. A disposable absorbent article in accordance with claim 13 wherein the absorbent panel has opposed longitudinally extending sides and each inner bond is inboard of a respective longitudinally extending side of the absorbent panel.

15. A disposable absorbent article in accordance with claim 13 wherein the absorbent panel has opposed longitudinally extending sides and each inner bond is outboard of a respective longitudinally extending side of the absorbent panel.

16. A disposable absorbent article in accordance with claim 13 wherein each sleeve extends longitudinally between first and second ends and a distal end of each inner gather is bonded, adjacent each of the first and second ends of the sleeve, to the topsheet inboard of the respective inner bond.

17. A disposable absorbent article in accordance with claim 13 wherein each sleeve extends longitudinally between first and second ends and the portion defining the outer gather is folded and bonded upon itself adjacent each of the first and second ends of the sleeve.

18. A disposable absorbent article in accordance with claim 5 wherein each sleeve has opposed longitudinally extending sides and the sleeve is bonded such that the opposed longitudinally extending sides overlap one another.

19. A disposable absorbent article in accordance with claim 18 wherein the longitudinally extending sides are positioned within longitudinally extending sides of the base portion.

20. A disposable absorbent article, comprising:
a backsheet;
an absorbent panel positioned on top of said backsheet;
a topsheet positioned on top of said absorbent panel;
a pair of longitudinally extending, elasticized leg cuff assemblies positioned on respective opposite sides of a longitudinal centerline of said article, wherein each said leg cuff assembly is configured to define an inner upstanding gather and an outer upstanding gather, each outer gather having an inner surface that is at an angle relative to a portion of the topsheet inboard of the outer gather that is less than or equal to about 90 degrees when the respective leg cuff assembly is in an initial stretched condition.

21. A method of forming a disposable absorbent article, comprising the steps of:
orienting a pair of longitudinally extending, elasticized leg cuff assemblies on respective opposite sides of a longitudinal centerline of the article;
defining on each of the leg cuff assemblies an inner upstanding gather having a first height relative to a topsheet of the article and an outer upstanding gather having a second height relative to the topsheet; and
maintaining the first height at approximately 65% or less than the second height.

22. A method of forming a disposable absorbent article, comprising the steps of:
orienting a pair of longitudinally extending, elasticized leg cuff assemblies on respective opposite sides of a longitudinal centerline of the article;
defining on each of the leg cuff assemblies an inner upstanding gather and an outer upstanding gather; and
maintaining each outer gather at an angle relative to a portion of a topsheet of the article inboard of the outer gather that is less than or equal to about 90 degrees when the respective leg cuff assembly is in an initial stretched condition.
